(19) United States
(12) Patent Application Publication
(10) Pub. No.: US 2026/0252734 A1
(43) Pub. Date: Aug. 27, 2026

(54) REIDENTIFYING PATIENTS ACROSS SITES IN A PRIVACY PRESERVING MANNER

(71) Applicant: Siemens Healthineers AG, Forchheim (DE)

(72) Inventors: Varghese KOLLERATHU, Whitefield (IN); Abhinandan TEJANI, Kudlu (IN); Matthias WOLF, Coatesville, PA (US)

(73) Assignee: Siemens Healthineers AG, Forchheim (DE)

(21) Appl. No.: 19/546,602

(22) Filed: Feb. 23, 2026

(30) Foreign Application Priority Data
Feb. 24, 2025 (DE) 10 2025 106 920.4

(51) Int. Cl. G06F 21/62; G06V 10/32; G06V 10/42; G06V 10/75; G06V 10/774; G06V 10/94; G16H 15/00

(52) U.S. Cl. CPC G06F 21/6254; G06V 10/32; G06V 10/42; G06V 10/753; G06V 10/774; G06V 10/945; G16H 15/00; G06V 2201/03

(57) ABSTRACT

A system for improving training data for training a machine learning model, comprises: a medical imaging unit to acquire a medical image of a current patient; a crossover detection unit to identify a patient crossover of the medical image and a previous dataset that is stored in a shared database and correlated to a previous patient; and a patient allocation unit to, in case the patient crossover is identified, allocate the medical image to one of a number of database cohorts according to the previous dataset for training the machine learning model.

### CROSS-REFERENCE TO RELATED APPLICATION(S)

The present application claims priority under 35 U.S.C. § 119 to German Patent Application No. 10 2025 106 920.4, filed Feb. 24, 2025, the entire contents of which is incorporated herein by reference.

### FIELD

One or more example embodiments of the present invention relate to a system for providing training data for training a machine learning model and a method for using the system.

### BACKGROUND

In order to train machine learning algorithms for analyzing medical images, it is common practice to divide available training data into multiple cohorts. For example, the available training data can be divided into at least training data, validation data, and testing data. It is further common practice to remove all private health information (PHI) by an anonymization tool in order to preserve data privacy. As a result, in case the same patient visits the same medical facility multiple times or in case the same patient visits multiple medical facilities whose medical data are merged onto a central location for training the machine learning algorithm, medical data of the same patient and, possibly, showing the same symptoms, may be assigned to different database cohorts-such as training data, validation data, and testing data-weakening the precision and/or the efficiency of the training and/or the validation of the machine learning algorithm. Independent of the grammatical term usage, individuals with male, female or other gender identities are included within the term. According to data available to the inventor, this so-called patient crossover can be estimated to be about 3%. In particular, the higher the density of medical facilities in a specific area, for example, at a metropolitan region, the higher the resultant patient crossover. Hence, there is still a need for providing a system for identifying the patent crossover while respecting data privacy matters to improve the training data.

### SUMMARY

It is an object of one or more example embodiments of the present invention to improve the training data for training the machine learning algorithm.

Accordingly, a system for improving training data for training a machine learning model is provided. The system comprising: a medical imaging unit for acquiring a medical image of a current patient; a crossover detection unit for identifying a patient crossover of the acquired medical image and a previous dataset stored in a shared database and correlated to a previous patient; and a patient allocation unit for, in case the patient crossover is identified, allocating the acquired medical image to one of a number of database cohorts according to the previous dataset for the training of the machine learning model.

The machine learning model is trained and/or will be trained for analyzing medical images. For example, the machine learning model can be trained and/or will be trained to identify anatomical structures or anomalies within medical images. The training of the machine learning model can be performed via supervised and/or unsupervised training. The present subject matter can be applied to any machine learning model used for analyzing medical images.

The medical imaging unit can be, for example, a computer tomography unit (CT), a magnetic resonance tomography unit (MRT), an X-ray device, or any other device capable of acquiring medical images. For the present case, a medical image can be understood as an illustration of anatomical and/or medical structures of a patient. For example, the medical image can be slices of a patient's body acquired by a CT unit.

The shared database can be located at the current medical facility, at another medical facility, and/or be stored on a cloud server. A dataset can be understood as a collection of data correlated to a medical examination, in particular, correlated to a single examination and/or to multiple examinations over time of a specific single patient. The dataset can include one or multiple medical images, findings correlated with the medical image or medical images and identification mechanism, device and/or means in order to correlate the data of the dataset with the specific patient. The shared database can include a single previous dataset or a set of previous datasets. In the latter case, the crossover detection unit can be configured to identify patient crossover of the acquired medical image and the set of previous datasets. Nonetheless, it is preferred that the crossover detection unit is configured to identify patient crossover of the acquired medical image with each previous dataset of the set of previous datasets at a time. In case no patient crossover is identified with a specific dataset of the set of previous datasets, a further previous dataset of the set of previous datasets can be used to identify patient crossover.

In the following, findings can be understood as medical and/or anatomical anomalies of a patient and/or diagnoses correlated to the medical and/or anatomical anomalies.

Landmarks can be understood as prominent locations within a medical image concerning the patient's body, for example, the top of the skull, the hepatic dome of the liver, the apex of the lung, or others. Landmarks can assist in determining the location and/or the size of the findings within the patient's body.

The previous patient can be the same specific person as the current patient or another patient. The term “previous” patient indicates that the previous patient is correlated to a previous dataset. In particular, a previous medical image and/or previous landmarks and/or findings of a previous dataset show and/or are correlated to the body of the previous patient. Furthermore, the term previous patient can represent multiple patients.

In the following, patient crossover can be understood as the circumstance of the same patient visiting the same medical facility multiple times and/or the same patient visiting multiple medical facilities whose medical data are merged, while the patient's medical data are assigned to different training cohorts for training and/or validating the machine learning algorithm. According to data available to the inventor, the patient crossover is estimated to be about 3%.

The database cohorts represent different groups of data in order to train the machine learning algorithm. The database cohorts comprise at least training data, validation data and testing data. For example, the training data can be used to train the machine learning algorithm for a specific purpose, for example, for identifying findings within medical images. The training data can be used during the learning process to fit parameters (e.g., weights) of, for example, a classifier. The validation data can be used to tune hyperparameters (i.e. the architecture) of the machine learning algorithm. The testing data can be used for testing purposes correlated with the trained machine learning algorithm. For example, the testing data can be used to assess the performance (i.e. generalization) of the trained machine learning algorithm. In case different medical images of the same person are used for different database cohorts, in particular when showing identical or similar findings and/or symptoms, the efficiency and/or the precision of the training of the machine learning algorithm can be violated. Furthermore, to use the same or similar data for training purposes and for validation and/or testing purposes can violate the principle of the validation and/or the testing of the machine learning algorithm.

In case patient crossover is identified, the acquired medical image is allocated to the one of the N2 database cohorts according to the previous dataset. Hence, in consequence, in case the previous dataset is allocated to the validation data, the acquired medical image is allocated to the validation data as well. This way, the identification of patient crossover can assist or ensure that all data, specifically all medical images correlated to the same patient, are allocated to the same database cohort. Therefore, the identification of patient crossover can increase the efficiency and/or the precision of the training of the machine learning algorithm.

According to an embodiment, the crossover detection unit is configured to identify the patient crossover by determining whether the current patient is identical to the previous patient. In particular, identity between the current patient and the previous patient occurs in case at least one of a number N1 of similarity checks indicates similarities between the acquired medical image and the previous dataset, with N1≥1. In particular, the N1 similarity checks comprise a first feature check, a second feature check, an image check, and/or a textual check.

The first feature check and/or the second feature check comprises an investigation whether higher order features correlated to the acquired medical image and the previous dataset are identical and/or show similarities. The image check comprises an investigation whether the acquired medical image shows optical and/or visual identity and/or similarities with the previous dataset. The textual check comprises an investigation whether a medical report based on the acquired medical image shows identity and/or similarities with another medical report based on the previous dataset. Alternatively, identity between the current patient and the previous patient can occur in case at least one of the N1 similarity checks indicates identity and/or similarities between a standardized medical image and the previous dataset.

According to a further embodiment, the crossover detection unit comprises a patient number allocation unit, wherein the patient number allocation unit is configured to generate a current patient number identifying the current patient and/or the a current medical facility of a number N2 of medical facilities, with N2≥1. In particular, the current patient number comprises an identification code identifying the current medical facility and/or an identification code identifying the current patient. In particular, the medical imaging unit is located at the current medical facility, wherein each of the N2 medical facilities is coupled with the shared database. In particular, the previous dataset is allocated to a previous medical image acquired at the current medical facility or at another one of the N2 medical facilities.

The patient number allocation unit can be configured to store the current patient number in an internal database of the system and/or in the shared database. In particular, the patient number allocation unit can be configured to store personal health information (PHI) correlated to the current patient in the internal database, for example, the patient's name, address, medical registration number, insurance company, and/or others. The data of the internal database is not to be shared with others of the N2 medical facilities whereas the N2 medical facilities are coupled to the data of the shared database. That is, the N2 medical facilities are in data connection and/or data exchange with the shared database. Hereby, medical facilities can be understood as hospitals, universities, institutions, and/or organizations performing medical examinations including the acquiring of medical images. Medical facilities can also be understood as departments and/or sub-units of the above-mentioned units. The previous medical image may have been acquired at any of the N2 medical facilities. In consequence, the previous dataset may have been generated at any of the N2 medical facilities. The previous medical image can be included within the previous dataset.

According to a further embodiment, the crossover detection unit comprises a standardizer unit, wherein the standardizer unit is configured to standardize the acquired medical image. In particular, the standardizer unit is configured to normalize the intensity and/or the amplitude of the acquired medical image to a given range, to crop the acquired medical image, to align the acquired medical image, to resample the acquired medical image, and/or to sub-sample the acquired medical image.

The standardization of the acquired medical image can also be understood as performing optical corrections to the acquired medical image. With the standardization, the fact that medical images can be acquired at different times, by different technicians, at different medical facilities, and/or by using different medical imaging units and/or different protocols can be compensated. The standardization can assist and/or ensure that data arising from multiple medical facilities are within the same permissible range. The characteristics relevant for identifying patient crossover are identical between the acquired medical image and the standardized medical image. Therefore, identifying a patient crossover of the standardized medical image has the same meaning as identifying a patient crossover of the acquired medical image.

According to a further embodiment, the system further comprises a user interface for receiving a current set of landmarks and/or findings correlated to the acquired medical image from a user of the system. In particular, the system further comprises an alignment unit for aligning the current set of landmarks and/or findings. In particular, the alignment unit is configured for calculating a distance and/or an affine transformation of the current set of landmarks and/or findings.

In Euclidean geometry, the affine transformation is a geometric transformation that preserves lines and parallelism, but not necessarily Euclidean distances and angles. Examples of the affine transformation include translation, scaling, homothety, similarity, reflection, rotation, hyperbolic rotation, shear mapping, and compositions of them in any combination and sequence. The alignment unit can be configured for adjusting the current set of landmarks and/or findings such a way that, for instance, the landmarks of different medical images are located at the same image location. In consequence, similar findings show congruence. This eases the comparison of findings for possible identity and/or temporal development. The user interface can be located at the medical imaging unit or at another location. The user interface can also be configured for receiving a command from the user for acquiring the medical image and/or a data unlearning command. The data unlearning command can be given in response to the wish of a specific patient to have all the data of the specific patient removed and/or not to be used for training the machine learning algorithm. The user interface can be a computer, tablet, mobile phone, and/or any other electronic device. The user interface can also be implemented as a speech recognition device. The user can be a technician and/or a physician. The user can directly interact with the user interface and/or interact with the user interface via a network connection and/or via the internet.

According to a further embodiment, the crossover detection unit comprises a transformation unit, wherein the transformation unit is configured to transform the standardized medical image to a private image portion and/or a shared image portion. In particular, the transformation unit is configured to transform the standardized medical image by performing a Fourier Analysis to derive phases and/or amplitudes, and/or by applying a trained transformation machine learning model. In particular, the derived phases form the shared image portion or the private image portion while the derived amplitudes form the respective other.

In other words, the transformation unit is configured to derive features from the standardized medical image. A part of said features forms the shared image portion while the remaining features form the private image portion. The transformation unit can be configured to store the private image portion in the internal database and/or to store the shared image portion in the shared database. By solely storing the shared image portion in the shared database, data privacy is kept. Only at the current medical facility, both the private image portion and the shared image portion are available, enabling the representation of the standardized medical image as a whole.

According to a further embodiment, the crossover detection unit comprises a volume generation unit, wherein the volume generation unit is configured to generate a first recon image by combining previous higher order features from the previous dataset with the current aligned set of landmarks and/or findings, and/or to generate a second recon image by combining current higher order features derived from the standardized medical image with a previous aligned set of landmarks and/or findings of the previous dataset.

The first recon image and/or the second recon image represents an artificially generated medical image based on a combination of given higher order features and a given set of landmarks and/or findings. Using said numerical data, the volume generation unit generates or reconstructs a visual medical image. Hereby, the volume generation unit can be configured to execute a Generative Algorithm such as Conditional GANs. Nonetheless, the volume generation unit can be configured to execute other algorithms as well. The previous higher order features and/or the previous aligned set of landmarks and/or findings have been extracted from the previous medical image, for example in the frame of a previous examination. The current higher order features are extracted from the standardized medical image while the current aligned set of landmarks and/or findings are correlated to the standardized medical image and are given by the user of the system, for example a technician and/or a physician.

According to a further embodiment, the crossover detection unit comprises a feature extraction unit for extracting higher order features from an input image. In particular, the feature extraction unit is configured to extract the higher order features by applying a transformation. In particular, the feature extraction unit is configured to extract the current higher order features from the standardized medical image, to extract first recon higher order features from the first recon image, and/or to extract second recon higher order features from the second recon image.

The feature extraction unit can be configured to apply a machine learning model, for example a deep learning model, that is trained on vast datasets that can be applied across a wide range of use cases. The deep learning model can be a foundation model. The foundation model can be an autoencoder. The autoencoder is a type of artificial neural network used to learn efficient coding of unlabeled data (unsupervised learning). The autoencoder learns two functions: an encoding function that transforms input data, and a decoding function that recreates the input data from the encoded representation. The feature extraction unit, in especially the autoencoder, learns an efficient representation of medical images for a dimensionality reduction of medical images. Hereby, lower-dimensional higher order features are generated based on the standardized medical image such as edges, patterns, colors, and/or the like.

According to a further embodiment, the crossover detection unit comprises a checking unit for performing the first feature check, the second feature check, the image check, and/or the textual check, wherein the first feature check comprises a comparison of the first recon higher order features with the current higher order features, and wherein the second feature check comprises a comparison of the second recon higher order features with the previous higher order features. In particular, the image check comprises a visual comparison of the standardized medical image with the first recon image. In particular, the crossover detection unit comprises a medical report generator for automatically generating a current medical report based on the standardized medical image and/or a recon medical report based on the first recon image, wherein the textual check comprises a comparison of the current medical report with the recon medical report.

Alternatively, or additionally, the image check can comprise a visual comparison of the standardized medical image with the second recon image. In a similar manner, the medical report generator can be configured for automatically generating the recon medical report based on the second recon image. The term “medical report” can be understood as a text-based collection and/or summary of the findings correlated to a given medical image, for example similar to the ones written by physicians and/or technicians. Hence, the medical report generator can be configured to automatically identify findings within the given medical image and to generate the text-based medical report including a classification and/or a specification of the findings concerning their location, size, meaning or others.

According to a further embodiment, the crossover detection unit is configured to iteratively select one of available datasets stored in the shared database as the previous dataset in order to identify the patient crossover of the standardized medical image with the available datasets. In particular, the crossover detection unit is configured to select only such of the available datasets which fulfill at least one of a number of selection criteria, wherein the selection criteria comprise a location threshold based on a spatial distance between the current medical facility and the one of the N2 medical facilities where the respective available dataset was generated, a landmarks threshold based on distances between landmarks of the current aligned set of landmarks and/or findings and landmarks of the respective available dataset, and/or a findings threshold based on proximities between findings of the current aligned set of landmarks and/or findings and findings of the respective available dataset.

Using said selection criteria, only those of the available datasets are chosen where patient crossover occurs with an increased chance. This way, the process of identifying patient crossover can be accelerated without a significant performance or precision reduction.

According to a further embodiment, the patient allocation unit is configured, in case the current patient is identical with the previous patient, to update the shared database by correlating the current patient number with a previous patient number stored in the previous dataset and to allocate the standardized medical image to one of the database cohorts according to the previous dataset, and/or, in case the current patient is not identical with the previous patient, to allocate the standardized medical image to one of the database cohorts based on a design choice and to update the shared database by creating a new dataset in the shared database including the current patient number, the shared portion of the standardized medical image, the current higher order features, and/or the current aligned set of landmarks and/or findings.

In other words, in case identity is found, the standardized medical image is assigned to the same one of the database cohorts the previous medical image stored in the previous dataset has been assigned to.

According to a further embodiment, the user interface is configured to receive a data unlearning command correlated to the current patient, and wherein the crossover detection unit is configured to unlearn all of the available datasets in the shared database showing a patient crossover with the current patient.

Hereby, the unlearning of data can be realized by deleting the data or by assigning the data with a respective marker, indicating that those data are not to be used for training the machine learning algorithm. The data unlearning command can be received from the user of the system, via the internet and/or via other networks.

According to a further embodiment, the system is configured to train the machine learning algorithm based on the available datasets in the shared database.

Hereby, the machine learning algorithm can be embodied as a deep learning algorithm or via other techniques. The training can be performed using the data of the shared database, in particular, using the available datasets. The training can be performed following a supervised and/or an unsupervised approach. With the system continuously and/or periodically identifying patient crossover and allocating the acquired medical image and/or the available datasets, the quality of the training is improved.

According to a further embodiment, the system is configured to use the trained machine learning algorithm, in particular, for detecting findings, anatomical structures and/or anomalies within the acquired medical image.

Anomalies can be, for example, nodes, blocked arteries, and/or other diseases. With the training of the machine learning algorithm being based on patient crossover-respecting training data, the usage of the trained machine learning algorithm can be more accurate and/or precise in identifying anomalies and/or anatomical structures within the acquired medical image of the current patient. In consequence, the treatment of the current patient can be improved and/or accelerated. In addition, technical and/or medical devices can be selected and/or used pinpointed according to the detected findings, anatomical structures and/or anomalies.

According to a second aspect, a method for using a system for providing training data for training a machine learning model is provided. The system comprises: a medical imaging unit, a crossover detection unit, and a patient allocation unit. In particular, the system is embodied according to the above-mentioned. The method comprises: acquiring a medical image of a current patient by the medical imaging unit; identifying a patient crossover of the acquired medical image with a previous dataset stored in a shared database correlated to a previous patient by the crossover detection unit; and in case the patient crossover is identified, allocating the acquired medical image to one of a number of database cohorts according to the previous dataset for the training of the machine learning model by the patient allocation unit.

The respective entity, e.g. the patient allocation unit, may be implemented in hardware and/or in software. If said entity is implemented in hardware, it may be embodied as a device, e.g. as a computer or as a processor or as a part of a system, e.g. a computer system. If said entity is implemented in software it may be embodied as a computer program product, as a function, as a routine, as a program code or as an executable object.

Any embodiment of the first aspect may be combined with any embodiment of the second aspect to obtain another embodiment of the second aspect.

According to a third aspect, one or more example embodiments of the present invention relate to a computer program product comprising a program code for executing the above-described method when run on at least one computer.

The embodiments and features described with reference to the suggested system apply mutatis mutandis to the suggested method and/or to the suggested computer program product.

Further possible implementations or alternative solutions of one or more example embodiments of the present invention also encompass combinations-that are not explicitly mentioned herein-of features described above or below with regard to the embodiments. The person skilled in the art may also add individual or isolated aspects and features to the most basic form of the present invention.

### BRIEF DESCRIPTION OF THE DRAWINGS

Further embodiments, features and advantages of the present invention will become apparent from the subsequent description and dependent claims, taken in conjunction with the accompanying drawings, in which:

FIG. 1 shows a schematical illustration of an embodiment of a system for providing training data for training a machine learning model;

FIG. 2 shows a schematical illustration of data flows occurring when using the system according to FIG. 1; and

FIG. 3 shows a schematical block diagram of an embodiment of a method for using the system according to FIG. 1.

In the Figures, like reference numerals designate like or functionally equivalent elements, unless otherwise indicated.

### DETAILED DESCRIPTION

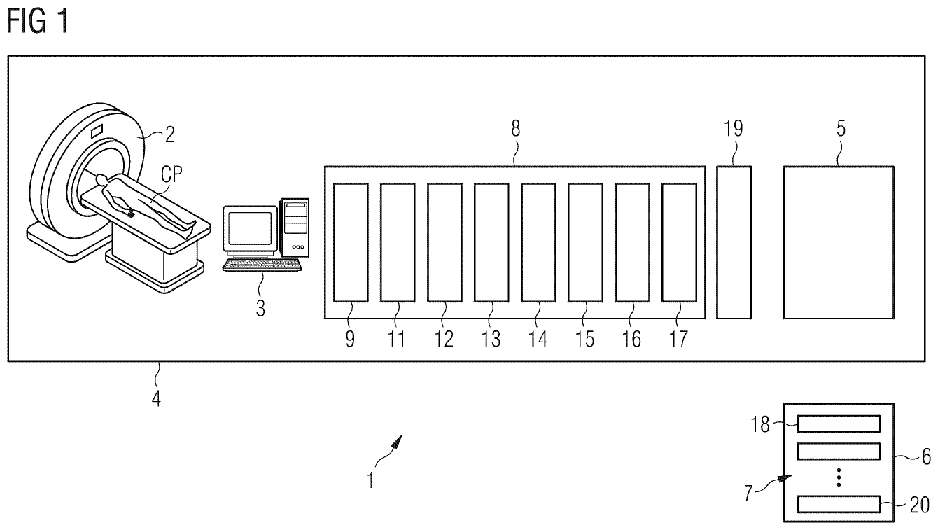

FIG. 1 shows a schematical illustration of an embodiment of a system 1 for improving not shown training data for training a not shown machine learning algorithm for analyzing medical images.

The system 1 comprises a medical imaging unit 2 for acquiring a medical image MI of a current patient CP. The medical imaging unit 2 may be, for example, a computer tomography device (CT), a magnetic resonance tomography (MRT), and an X-ray device, or the like. The acquired medical image MI may show, for example, one or more sections or slices of the current patient CP. The system 1 further comprises a user interface 3 for receiving user commands of a not shown user of the system 1. The user commands can comprise a command for acquiring the medical image MI, for detecting a possible patient crossover, for unlearning data correlated to the current patient CP, or others. The user interface 3 is further configured to receive a current set of landmarks and/or findings CL correlated to the acquired medical image MI. Landmarks can be, for example, the top of the skull, the hepatic dome of the liver, the apex of the lung, or others. The landmarks are used to specify the location of findings inside the body of a patient. Findings can be, for example, medical structures and/or anomalies, for example nodes. The current set of landmarks and/or findings CL can be specified by the user of the system 1, for example a physician or technician. The medical imaging unit 2 and the user interface 3 are located at a current medical facility 4. The current medical facility 4 can be a hospital, a university, a respective department of the hospital or the university, or others. The system 1 can be located at the current medical facility 4, at a further not shown medical facility, and/or be implemented on a not shown cloud server.

The system 1 further comprises an internal database 5 being located at the current medical facility 4. The internal database 5 is used to save all private health information (PHI) of the current patient CP, for example, the name of the current patient CP, the medical registration number (MRM) of the current patient CP, or others. Due to data privacy reasons, said data are not to be shared outside the current medical facility 4. The internal database 5 may be a hard drive and/or a server located at the current medical facility 4.

The system 1 further comprises a shared database 6 which can be stored at the current medical facility 4, at another not displayed medical facility, stored in a cloud server, and/or at other places. The shared database 6 comprises a number of available datasets 7 which can be used for training the machine learning algorithm. The available datasets 7 can be accessed by any medical facility, including the current medical facility 4. In other words, the shared database 6 is used to exchange data between all of the medical facilities.

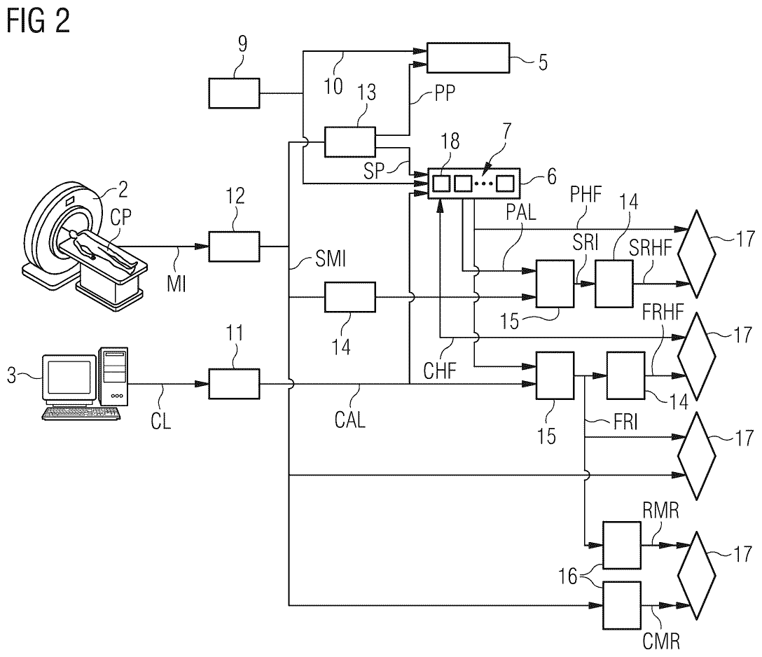

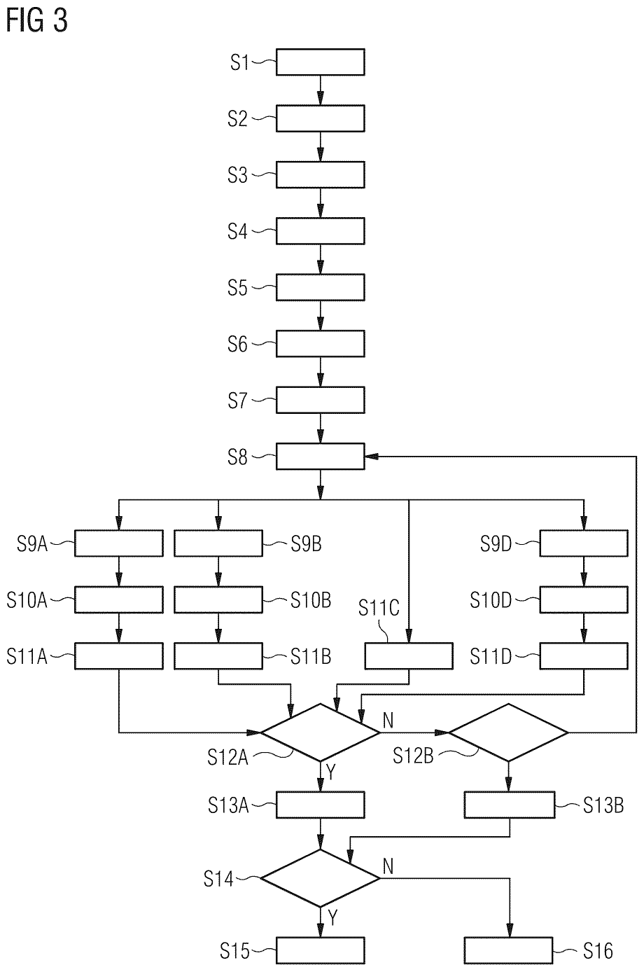

The system 1 further comprises a crossover detection unit 8 for detecting a possible patient crossover between the acquired medical image MI and the available datasets 7 stored in the shared database 6. In the following, the components of the crossover detection unit 8 are explained with reference to FIG. 1. A data flow generated by the crossover detection unit 8 in order to identify the patient crossover will be explained in detail with reference to FIG. 2. In FIG. 2, multiple representations of features do not indicate a multiple existence of the features, but multiple usages of the same feature. In the following, the FIGS. 1 and 2 will be referred to at the same time. The individual steps performed by the crossover detection unit 8 will be explained in detail with reference to the block diagram displayed in FIG. 3 below.

The crossover detection unit 8 comprises a patient number allocation unit 9. The patient number allocation unit 9 is configured to identify whether the current patient CP has already been examined at the current medical facility 4. For that purpose, the patient number allocation unit 9 is configured to access the internal database 5 for detecting previous examinations of the current patient CP. In case the current patient CP has already been examined at the current medical facility 4, the acquired medical image MI is assigned to the same one of the database cohorts as in the frame of the previous examinations of the current patient CP. In case the current patient CP is novel to the current medical facility 4, there still can exist previous examinations of the current patient CP at other medical facilities. In order to detect such patient crossover, the patient number allocation unit 9 is configured to generate a current patient number 10 (see FIG. 2) which comprises a signature for identifying the current medical facility 4 and a signature for identifying the current patient CP. For example, the current patient number 10 may be “1-276” with the digit “1” identifying the current medical facility 4 and the digits “276” identifying the current patient CP. The current patient number 10 is not correlated to the medical registration number of the current patient CP or other official numbers covered by data privacy matters. The patient number allocation unit 9 is configured to store the current patient number 10 in the internal database 5 together with private health information (PHI) of the current patient CP like the name of the current patient CP, the medical registration number, or others. In addition, the patient number allocation unit 9 is configured to store the current patient number 10 in the shared database 6 (see FIG. 2).

The crossover detection unit 8 further comprises an alignment unit 11 for aligning the current set of landmarks and/or findings CL. The alignment comprises the calculation of distances of the respective landmarks visible in the acquired medical image MI and the calculation of a respective affine transformation. Alternatively, or additionally, the alignment comprises the calculation of distances of the respective findings correlated to the acquired medical image MI as well as the calculation of the affine transformation of the respective findings. The purpose of the alignment is to convert the locations and/or sizes of the landmarks and/or findings in a numerical manner for further numerical processing of said data, including their comparison with other data stored in the shared database 6. In the following, the aligned current set of landmarks and/or findings CL will be referred to as the current aligned set of landmarks and/or findings CAL.

The crossover detection unit 8 further comprises a standardizer unit 12 which is configured to standardize the acquired medical image MI. The standardization comprises, for example, to normalize the acquired medical image MI to a certain range. It can include data processing steps like intensity and/or amplitude normalization, a cropping, a resampling, a subsampling of the acquired medical image MI, or others. The purpose of the standardization is to pay respect to the effect that similar medical images may vary in intensity or other parameters. Furthermore, for each examination with the medical imaging unit 2, the patients may be positioned in a different way. In the following, the output of the standardizer unit 12 is referred to as the standardized medical image SMI.

The crossover detection unit 8 further comprises a transformation unit 13. The transformation unit 13 is configured to transform the standardized medical image SMI to a private image portion PP which is stored in the internal database 5 and a shared image portion SP which is stored in the shared database 6. The transformation unit 13 can transform the standardized medical image SMI by performing a Fourier analysis in order to derive phases and amplitudes and/or by applying a transformation machine learning model. The derived phases can form the shared image portion SP while the derived amplitudes can form the private image portion PP. Nonetheless, said assignment can be set inverse, as well. By storing only one image portion of the standardized medical image SMI on the shared database 6, data privacy is respected.

The crossover detection unit 8 further comprises a feature extraction unit 14. The feature extraction unit 14 is configured for extracting higher order features from a given input image. Hereby, the feature extraction unit 14 is configured to extract the higher order features by applying a foundation model, in particular an autoencoder. Said higher order features can comprise, for example, edges, patterns, colors, textures, and/or other elements of the given input image. The feature extraction unit 14 is configured to extract a number of current higher order features CHF from the standardized medical image SMI. The extracted current higher order features CHF are stored in the shared database 6.

The crossover detection unit 8 further comprises a volume generation unit 15. The volume generation unit 15 can perform a generative algorithm such as conditional GANs to generate an artificial visual medical image based on given numerical data, in especially based on a combination of given higher order features and landmarks and/or findings. In a simplified way it can be said that the volume generation unit 15 is configured to derive information concerning which elements can be seen inside the generated medical image from the higher order features, while it derives information concerning at which location inside the generated medical image said elements are located from the landmarks and/or findings.

The crossover detection unit 8 further comprises a medical report generator 16 for automatically generating a text-based medical report based on a given medical image. In particular, the medical report generator 16 is configured to automatically identify findings within the given medical image and to generate the text-based medical report including a classification and/or a specification of the findings concerning their location, size, meaning or others.

The crossover detection unit 8 further comprises a checking unit 17 for performing a comparison between two or more input data and an identification of similarities between the input data. Hereby, the input data can be higher-order features, medical images, and/or text-based medical reports.

For identifying a patient crossover, the crossover detection unit 8 is configured to select one of the available datasets 7 of the shared database 6. Hereby, the crossover detection unit 8 can be configured to select only such of the available datasets 7 which fulfill at least one of a number of selection criteria. Said selection criteria can comprise a location threshold based on a spatial distance between the current medical facility 4 and that very medical facility, where the respective available dataset 7 was generated. In other words, only such of the available datasets 7 can be selected which were generated in vicinity to the current medical facility 4. The selection criteria may further comprise a landmark threshold based on distances between the landmarks of the current aligned set of landmarks and/or findings CAL and landmarks of the respective available dataset 7. Furthermore, the selection criteria can comprise a finding threshold based on proximities between findings of the current aligned set of landmarks and/or findings CAL and findings of the respective available dataset 7. The purpose of the selection criteria is to accelerate the identification of patient crossover by focusing on comparing the standardized medical image SMI with only such available datasets 7 where a possible patient crossover is likely to occur. Then, the standardized medical image SMI is iteratively compared to all selected of the available datasets 7, hence one after the other. In the following, the respective selected one of the available datasets 7 will be referred to as the previous dataset 18.

After selecting the previous dataset 18, the volume generation unit 15 is configured to load previous higher order features PHF stored in the previous dataset 18. In addition, the volume generation unit 15 is configured to load the current aligned set of landmarks and/or findings CAL. The volume generation unit 15 is further configured to generate an artificial medical image based on the loaded previous higher order features PHF and the current aligned set of landmarks and/or findings CAL. The generated medical image is referred to as a first recon image FRI. The feature extraction unit 14 is configured to extract first recon higher order features FRHF of the generated first recon image FRI. Then, the checking unit 17 is configured to compare the first recon higher order features FRHF with the current higher order features CHF.

At the same time or sequentially, the volume generation unit 15 is configured to load the previous aligned set of landmarks and/or findings PAL stored in the previous dataset 18. Furthermore, the volume generation unit 15 is configured to load the current higher order features CHF. Then, the volume generation unit 15 is configured to generate a further artificial medical image based on the previous aligned set of landmarks and/or findings PAL and the current higher order features CHF. The further generated medical image is referred to as a second recon image SRI. Consequently, the feature extraction unit 14 is configured to extract second recon higher order features SRHF of the second recon image SRI. Then, the checking unit 17 is configured to compare the second recon higher order features SRHF with the previous higher order features PHF.

At the same time, sequentially, or in case the previous two comparisons of the checking unit 17 show a similarity, the checking unit 17 is configured to compare the standardized medical image SMI with the first recon image FRI.

At the same time, sequentially, or in case the previous three comparisons of the checking unit 17 show a similarity, the medical report generator 16 is configured to automatically generate a current medical report CMR based on the standardized medical image SMI. Furthermore, the medical report generator 16 is configured to automatically generate a recon medical report RMR based on the first recon image FRI. Then, the checking unit 17 is configured to compare the current medical report CMR with the recon medical report RMR.

In case at least one, two, three, or all of the performed comparisons performed by the checking unit 17 show a similarity, the standardized medical image SMI is considered to represent a patient crossover with the previous dataset 18. In consequence, the current patient CP is considered to be identical with a not shown previous patient correlated to the previous dataset 18.

The system 1 further comprises a patient allocation unit 19. The patient allocation unit 19 is configured, in case the current patient CP is considered to be identical with the previous patient, to update the shared database 6 by correlating the current patient number 10 with a not shown previous patient number correlated to the previous patient and/or stored in the previous dataset 18, to add the current patient number 10 to the previous dataset 18, and to allocate the standardized medical image SMI to one of the database cohorts according to the previous dataset 18. In other words, in case the previous dataset 18 is allocated to the validation data, the standardized medical image SMI is allocated to the validation data as well. Alternatively, or additionally, the patient allocation unit 19 is configured to, in case the current patient CP is not identical with the previous patient, to update the shared database 6 by creating a new dataset 20 within the shared database 6 including the current patient number 10, the shared image portion SP of the standardized medical image SMI, the current aligned higher order features CHF, and/or the current aligned set of landmarks and/or findings CAL. Furthermore, patient allocation unit 19 is configured to allocate the standardized medical image SMI to one of the database cohorts based on a given design choice for training the machine learning algorithm.

The user interface 3 can be configured to receive a not shown data unlearning command. Upon receiving said data unlearning command, the crossover detection unit 8 is configured to identify all patient crossovers of the respective patient requesting the data unlearning command with all available datasets 7 in the shared database 6. Then, all identified ones of the available datasets 7 showing a patient crossover with the respective patient can either be assigned with a marker in order to not use the respective available datasets 7 for training the machine learning algorithm, and/or the identified datasets can be deleted from the shared database 6. This way, it can be ensured that the respective patient data is not used for training the machine learning algorithm not only at the current medical facility 4, but at all medical facilities accessing the shared database 6. Hereby, established unlearning techniques like shared, isolated, sliced and aggregated training may be utilized at the medical facilities to unlearn the respective patient data.

FIG. 3 shows a schematical block diagram for using the system 1.

In a first step S1, the current patient number 10 is generated by the patient number allocation unit 9. In a further step S2, the medical image MI is acquired by the medical imaging unit 2. In a further step S3, the current set of landmarks and/or findings CL is received by the user interface 3. In a further step S4, the current set of landmarks and/or findings CL is aligned by the alignment unit 11 to form the current aligned set of landmarks and/or findings CAL. In a further step S5, the acquired medical image MI is standardized by the standardizer unit 12 to form the standardized medical image SMI. In a further Step S6, the standardized medical image SMI is transformed by the transformation unit 13 to form the private image portion PP and the shared image portion SP. In a further step S7, the current higher order features CAF are extracted by the feature extraction unit 14.

### In a Further Step S8, One of the Available Datasets 7

stored on the shared database 6 is selected to form the previous dataset 18. In addition, the previous aligned set of landmarks and/or findings PAL and previous higher order features PHF are loaded from the selected previous dataset 18.

In a further step S9A, the first recon image FRI is generated based on the current aligned set of landmarks and/or findings CAL and the previous higher order features PHF by the volume generation unit 15. In a further step S10A, the first recon higher order features FRHF are extracted from the first recon image FRI by the feature extraction unit 14. In a further step S11A, the first feature check is performed by comparing the first recon higher order features FRHF with the current higher order features CHF by the checking unit 17.

In a further step S9B, the second recon image SRI is generated based on the previous aligned set of landmarks and/or findings PAL and the current higher order features CHF by the volume generation unit 15. In a further step S10B, the second recon higher order features SRHF are extracted from the second recon image SRI by the feature extraction unit 14. In a further step S11B, the second feature check is performed by comparing the previous higher order features PHF with the second recon higher order features SRHF by the checking unit 17.

In a further step S11C, the image check is performed by comparing the standardized medical image SMI and the first recon image FRI by the checking unit 17.

In a further step S9D, the current medical report CMR is automatically generated based on the standardized medical image SMI by the medical report generator 16. In a further step S10D, the recon medical report RMR is automatically generated based on the first recon image FRI by the medical report generator 16. In a further step S11D, the textual check is performed by comparing the current medical record CMR with the recon medical report RMR by the checking unit 17.

Hereby, the above-mentioned four method branches S9A-S11A, S9B-S11B, S11C, and/or S9D-S11D can be performed at the same time or after one another.

In a further step S12A, the results of the first feature check S11A, the second feature check S11B, the image check S11C and/or the textual check S11D are analyzed to determine a possible patient crossover of the standardized medical image SMI with the previous dataset 18 by the checking unit 17. In case a patient crossover is identified, in a further step S13A, the shared database 6 is updated by adding the current patient number 10 to the previous dataset 18. The method then continues with a step S14 which will be explained below. In case a patient crossover of the standardized medical image SMI with the previous dataset 18 is not identified, in a step S12B it is determined whether a further one of the available datasets 7 can be analyzed for a possible patient crossover with the standardized medical image SMI. If yes, then the previous routine is repeated beginning with the step S8 and a possible patient crossover between the standardized medical image SMI and the further one of the available datasets 7 is analyzed. In case all of the available datasets 7 have been analyzed for a possible patient crossover, hereby possibly respecting the selection criteria, in a further step S13B, the new dataset 20 is created in the shared database 6.

In the step S14, the system 1 checks whether the data unlearning command has been entered by the user interface 3. In case the data unlearning command has been given, the unlearning of the patient data from the shared database 6 is performed in a step S15. Otherwise, in a further step S16, the machine learning algorithm is trained using all available datasets 7 on the shared database 6.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although Described With Reference to Specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been described in accordance with preferred embodiments, it is obvious for the person skilled in the art that modifications are possible in all embodiments.

### REFERENCE NUMERALS

1 system
2 medical imaging unit
3 user interface
4 current medical facility
5 internal database
6 shared database
7 available datasets
8 crossover detection unit
9 patient number allocation unit
10 current patient number
11 alignment unit
12 standardizer unit
13 transformation unit
14 feature extraction unit
15 volume generation unit
16 medical report generator
17 checking unit
18 previous dataset
19 patient allocation unit
20 new dataset
CAL current aligned set of landmarks and/or findings
CHF current higher order features
CL current set of landmarks and/or findings
CP current patient
FRHF first recon higher order features
FRI first recon image
MI medical image
PAL previous aligned set of landmarks and/or findings
PHF previous higher order features
PP private image portion
RMR recon medical report
SMI standardized medical image
S1-S16 step
SP shared image portion
SRHF second recon higher order features
SRI second recon image

What is claimed is:

1. A system for improving training data for training a machine learning model, the system comprising:
a medical imaging unit configured to acquire a medical image of a current patient;
a crossover detection unit configured to identify a patient crossover of the medical image and a previous dataset, the previous dataset stored in a shared database and correlated to a previous patient; and
a patient allocation unit configured to, in case the patient crossover is identified, allocate the medical image to one of a number of database cohorts according to the previous dataset, for training the machine learning model.

2. The system according to claim 1, wherein the crossover detection unit is configured to identify the patient crossover by determining whether the current patient is identical to the previous patient.

3. The system according to claim 1, wherein the crossover detection unit comprises:
a patient number allocation unit configured to generate a current patient number identifying at least one of the current patient or a current medical facility of a number of medical facilities, wherein the number of medical facilities is greater than or equal to one.

4. The system according to claim 3, wherein the crossover detection unit comprises:
a standardizer unit configured to standardize the medical image by at least one of
normalizing at least one of an intensity or an amplitude of the medical image to a given range,
cropping the medical image,
aligning the medical image,
resampling the medical image, or
sub-sampling the medical image.

5. The system according to claim 3, further comprising:
a user interface configured to receive, from a user, a current set of at least one of landmarks or findings correlated to the medical image.

6. The system according to claim 4, wherein the crossover detection unit comprises:
a transformation unit configured to transform the standardized medical image to at least one of a private image portion or a shared image portion by at least one of
performing a Fourier Analysis to derive at least one of phases or amplitudes, or
applying a trained transformation machine learning model, wherein
derived phases form one of the shared image portion or the private image portion while derived amplitudes form another of the shared image portion or the private image portion.

7. The system according to claim 5, wherein the crossover detection unit comprises:
a volume generation unit configured to at least one of
generate a first recon image by combining previous higher order features from the previous dataset with a current aligned set of at least one of landmarks or findings, or
generate a second recon image by combining current higher order features derived from a standardized medical image with a previous aligned set of at least one of landmarks or findings of the previous dataset.

8. The system according to claim 7, wherein the crossover detection unit comprises:
a feature extraction unit configured to extract higher order features from an input image by applying a transformation.

9. The system according to claim 8, wherein the crossover detection unit comprises:
a checking unit configured to perform at least one of a first feature check, a second feature check, an image check, or a textual check, wherein
the first feature check includes a comparison of first recon higher order features with the current higher order features,
the second feature check includes a comparison of second recon higher order features with the previous higher order features,
the image check includes a comparison of the standardized medical image with the first recon image,
the crossover detection unit includes a medical report generator configured to automatically generate at least one of a current medical report based on the standardized medical image or a recon medical report based on the first recon image, and
the textual check includes a comparison of the current medical report with the recon medical report.

10. The system according to claim 5, wherein the crossover detection unit is configured to iteratively select one of available datasets stored in the shared database as the previous dataset in order to identify the patient crossover of a standardized medical image with the available datasets.

11. The system according to claim 7, wherein at least one of
the patient allocation unit is configured to, in case the current patient is identical with the previous patient,
update the shared database by correlating the current patient number with a previous patient number stored in the previous dataset, and
allocate the standardized medical image to one of the number of database cohorts according to the previous dataset, or
the patient allocation unit is configured to, in case the current patient is not identical with the previous patient,
allocate the standardized medical image to one of the number of database cohorts based on a design choice, and
update the shared database by creating a new dataset in the shared database including at least one of the current patient number, a shared portion of the standardized medical image, the current higher order features, or the current aligned set of at least one of landmarks or findings.

12. The system according to claim 10, wherein
the user interface is configured to receive a data unlearning command correlated to the current patient, and
the crossover detection unit is configured to unlearn all of the available datasets in the shared database showing a patient crossover with the current patient.

13. The system according to claim 1, wherein the system is configured to train a machine learning algorithm based on available datasets in the shared database.

14. The system according to claim 13, wherein the system is configured to use the machine learning algorithm to detect at least one of findings, anatomical structures or anomalies within the medical image.

15. A method for using the system of claim 1 to provide training data for training a machine learning model, the method comprising:
acquiring, by the medical imaging unit, the medical image of the current patient;
identifying, by the crossover detection unit, the patient crossover of the medical image with the previous dataset stored in the shared database correlated to the previous patient; and
in case the patient crossover is identified, allocating by the patient allocation unit, the medical image to one of the number of database cohorts according to the previous dataset for training the machine learning model.

16. The system according to claim 2, wherein identity between the current patient and the previous patient occurs in case at least one of a number of similarity checks indicates similarities between the medical image and the previous dataset, wherein the number of similarity checks is greater than or equal to one.

17. The system according to claim 16, wherein the number of similarity checks include at least one of a first feature check, a second feature check, an image check, or a textual check.

18. The system according to claim 3, wherein
the current patient number includes at least one of an identification code identifying the current medical facility or an identification code identifying the current patient.

19. The system according to claim 18, wherein
the medical imaging unit is located at the current medical facility, and
each of the number of medical facilities is coupled with the shared database.

20. The system according to claim 19, wherein the previous dataset is allocated to a previous medical image acquired at the current medical facility or at another one of the number of medical facilities.

21. The system according to claim 5, further comprising:
an alignment unit configured to
align the current set of at least one of landmarks or findings, and
calculate at least one of a distance or an affine transformation of the current set of at least one of landmarks or findings.

22. The system according to claim 8, wherein the feature extraction unit is configured to at least one of
extract the current higher order features from the standardized medical image,
extract first recon higher order features from the first recon image, or
extract second recon higher order features from the second recon image.

23. The system according to claim 10, wherein
the crossover detection unit is configured to select only available datasets that fulfill at least one of a number of selection criteria, and
the number of selection criteria include at least one of
a location threshold based on a spatial distance between the current medical facility and a medical facility of the number of medical facilities where a respective available dataset was generated,
a landmarks threshold based on at least one of distances between landmarks of a current aligned set of landmarks or distances between findings and landmarks of the respective available dataset, or
a findings threshold based on proximities between findings of a current aligned set of at least one of landmarks or findings and findings of the respective available dataset.
